(19) United States
(12) Patent Application Publication
(10) Pub. No.: US 2009/0192445 A1
(43) Pub. Date: Jul. 30, 2009

(54) Microdialysis Probe

(75) Inventor: Henrik Jessen, Haninge (SE)

Correspondence Address: SCHWEGMAN, LUNDBERG & WOESSNER, P.A., P.O. BOX 2938, MINNEAPOLIS, MN, 55402, US

(73) Assignee: CMA/MICRODIALYSIS AB, Solna (SE)

(21) Appl. No.: 12/083,956

(22) PCT Filed: Oct. 24, 2006
(86) PCT No.: PCT/EP2006/067710

§ 371 Date: Mar. 12, 2009

Related U.S. Application Data
(60) Provisional application No. 60/596,974, filed on Nov. 2, 2005.

(30) Foreign Application Priority Data
Oct. 27, 2005 (SE) 0502390-8

(51) Int. Cl. A61M 25/14

(52) U.S. Cl. 604/27

(57) ABSTRACT

The invention relates to an improved linear microdialysis probe comprising a continuous length of flexible tubing (1) having at least one window (4) formed therein, said window covering at least one part of the circumference of the tubing, while the remaining part forms at least one unbroken connection between a first end of said tubing and a second end of said tubing, said ends adapted to be attached to an inlet for perfusion liquid and the other end forming an outlet for the dialysate, said at least one window (4) exposing a tubular semipermeable membrane (2).

The present invention relates to an improved linear microdialysis probe for use in medical devices, more specifically for use for diagnostic or testing purposes.

The meaning of specific wordings in the text should be interpreted as follows:

The word probe should be interpreted also as catheter.

The inlet and outlet of the probe as described may in case of a reversed flow be used as outlet and inlet, respectively.

Perfusion liquid is the liquid used in the microdialysis, which is allowed to enter the probe and there take up substances from the surrounding tissue through a membrane.

The perfusion liquid becomes the dialysate after the dialysis.

### BACKGROUND OF THE INVENTION

Microdialysis is a method of examination in which a probe is inserted into tissue in vivo, such that one side of a semi-permeable membrane is in contact with tissue and extra cellular liquid and the other side is flushed or rinsed with a dialysis liquid (perfusate) which takes up substances from the extra cellular liquid through the membrane. A substance can also be distributed locally to the extra-cellular liquid through the perfusion liquid. These substances can then be analyzed in the dialysate on or after exiting the probe.

Microdialysis probes are by nature fragile and very small, which requires great care in inserting and withdrawing the probe from the tissue in which it is used. Probes are often made in the form of an inner and an outer tube, where the outer tube exhibits a membrane and the dialysate and the perfusate is entering and exiting the tube at one end and the other end of the tubes are fused or plugged. The rigid or flexible type cannula probe have, even though their geometries differ, relatively large diameters at the junction of their inlet and outlet tubing since they enter and exit a body at the same point. This type of probe is usually entered into brain tissue or some other tissue from the outside of the body and thus also has a limited available depth depending on the length of the probe, where the dialysis may take place. This design also has limitations in the attachment and insertion of the probe to/in soft and/or moving tissue, e.g. skin, heart, liver, eye, tumor.

Such microdialysis probes of this kind are described in SE-C-434 214, U.S. Pat. No. 5,735,832 and U.S. Pat. No. 5,741,284.

It is a fact that microdialysis provides a unique possibility to examine the equilibrias of substances and/or the amounts present or missing of substances or to monitor specific changes in the status of substances connected with e.g. the use of medicaments, in surgery etc. However, with the probes presented in the prior art the possibilities of performing dialysis in e.g. the dermis or for that matter, in other tissue, such as liver, heart, or tumors etc., which generally cannot be reached, or as regards the dermis no possibility exists of using the common type of probe, are limited. For this type of dialysis a linear probe would be more satisfactory. An example of this type of probe is known from U.S. Pat. No. 5,706,806. However, there is a problem related to such linear probes as they are thin and the tubing making up the probe is thin there will be a certain amount of strain on the probe during insertion, use and extraction as the membrane part will inevitable make the probe prone to rupture.

The linear probe described in U.S. Pat. No. 5,706,806 comprises a probe assembly consisting of two long lengths of plastic tubing having a short, thin semipermeable membrane window joining the tubing parts. The tubing also contains a length of strong but flexible, inert reinforcement or support fiber, preferably extending though the whole length of the tubing, but at least extending past the window and out beyond one end of the tubing. The support fiber is also attached or bonded to the other end of the tubing to help resist longitudinal stress when pulling the probe through tissue. The support fiber and the plastic tubing are sealed at the ends, to be cut off after insertion. The support fiber by nature will be an object resisting the free flow of the dialysis liquid through the probe.

### SUMMARY OF THE INVENTION

When using the common microdialysis probe there are limitations to the use of the same as the probe has a defined length which limits the depth of invasion into the body or organ. The length is in turn limited by considerations of the ability to withstand forces form the surrounding tissue, e.g. a muscle. In the case of an organ not easily accessible from the outside of the body there will exist a problem of inserting and keeping the probe in the place of dialysis. Also if the probe is used deep into any tissue an opening may have to been kept open in order to the probe to be inserted and retracted.

The solution to the problem is then to use a linear probe as described above, but a linear probe according to the invention which does not have any of the draw-backs which are present in the linear probes according to the prior art.

It is thus an object of the invention to provide a microdialysis probe, which is flexible and which may be inserted both into the skin and also in organs more deeply located, such that the common type of probe will be difficult to attach and remain attached during the microdialysis procedure.

It is thus a further object of the invention to provide a microdialysis probe allowing a flow free from unnecessary obstacles in the flow path while still retaining the original strength of the probe.

It is also an object of the invention to provide a microdialysis probe, which is suitable for the general use in living tissue when taking samples for e.g. diagnostic purposes.

These objects are attained by the microdialysis probe according to the invention in which the linear microdialysis probe comprises a continuous length of flexible tubing having at least one window formed therein, said window covering at least one part of the circumference of the tubing, while the remaining part/-s form at least one unbroken connection between a first end of said tubing and a second end of said tubing, said ends adapted to be attached to an inlet for perfusion liquid and the other end forming an outlet for the dialysate, said at least one window exposing a tubular semipermeable membrane.

There could of course be more than one window arranged.

Further objects of the invention are attained by the microdialysis probe according to the invention in which the tubular semipermeable membrane is arranged inside the flexible tubing and sealed thereto.

In accordance with the invention, these and other objects evident from the description of the invention are accomplished in a microdialysis probe according to the invention and as claimed in the appended claims.

### BRIEF DESCRIPTION OF THE DRAWINGS

These and other objects, advantages and features of the present invention will be more readily understood from the following detailed description of the preferred embodiments thereof, when considered in conjunction with the drawings, in which like reference numerals indicate identical structures throughout the several views, and wherein:

FIG. 1 shows a generalized side view of the linear probe according to the invention;

FIGS. 2a and 2b show longitudinal sectional view along A-A in FIG. 3a and along B-B of the linear probe according to the invention showing the inserted and fixed membrane.

FIGS. 3a and 3b show cross sections through the probe at a point where the membrane partly covers the circumference of the probe

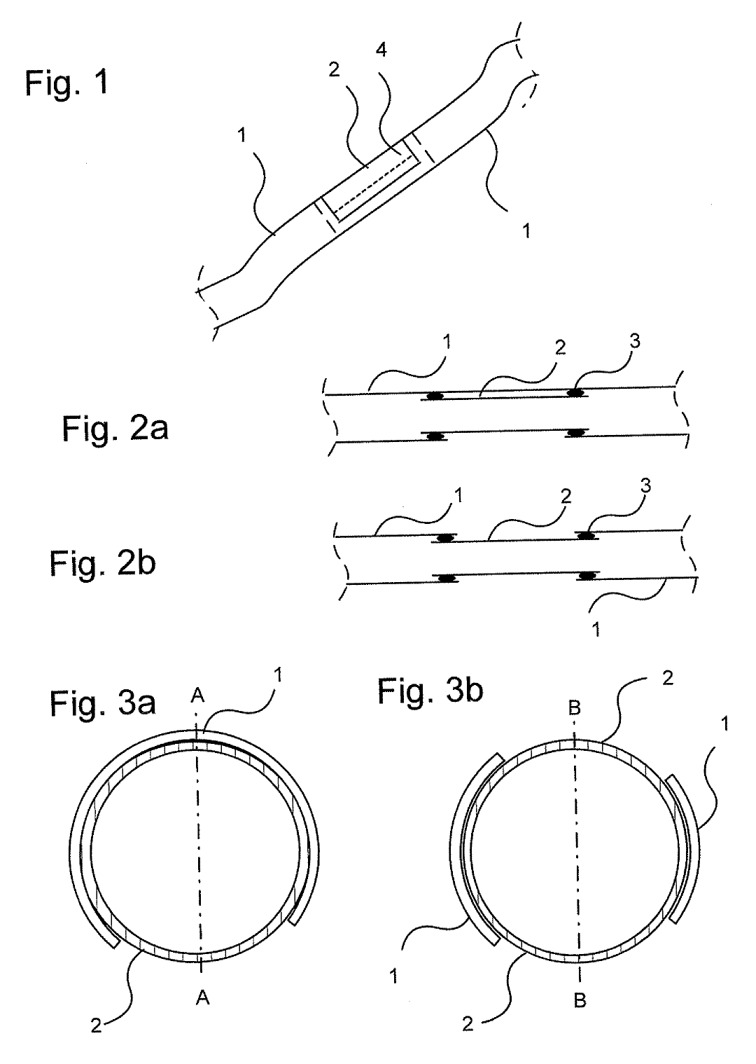

In FIG. 1 the probe is shown in a side view where the parts denoted 1 is the probe tube, i.e. a long flexible plastic tubing in which a window 4 has been cut in order to provide a space for the semipermeable membrane 2 which is arranged on the inside of the tubing. Suitable semipermeable membranes are well known within the art and include e.g. polyacrylonitrile, cuprophan, polysulphone, or the like. The material of the plastic tubing is e.g. polyimide and the thickness of the tube walls is in the interval approx. 4.48-5.68 μm and may be purchased as polyimidetubing. The semi-permeable membrane may be made from e.g. Cuprophane®. The measurements are examples only and should not be limiting to the invention

In FIGS. 2a and 2b is shown how the membrane 2 is fitted into the flexible tubing 1 and glue 3 is used for attaching the membrane to the tubing. The manner of providing the membrane in the tubing is to cut open a window in the tubing and arrange the membrane inside the tubing and apply glue around the inner perimeter of the tubing. As may be gained from the figures the membrane is also tubular which gives a better fastening of the membrane 2 to the tubing 1 and ascertains that there will be no leakage between the tubing and the membrane.

In FIGS. 3a and 3b a cross section is shown through the probe at the area of the membrane. In FIG. 3a is shown one window exposing the membrane 2 to the contact with tissue to be tested and in FIG. 3b two windows are show for the same purpose.

In a further embodiment windows in the form of holes may be made e.g. using a laser. These holes may be arranged around the perimeter of the tube grouped e.g. in the form of a spiral. The membrane is then arranged inside the tube giving the perfusate access to the surrounding tissue in order for a microdialysis to take place.

In order to introduce the probe at the location where the dialysis is to take place a cannula is inserted into the tissue wherein the membrane of the probe is to be situated. The (outlet) end of the tubing is then entered into the cannula and is guided through the tip of the cannula and pulled forward until the membrane is in its intended position. The cannula is thereafter withdrawn in the opposite direction to the direction of insertion of the cannula. Meanwhile the tubing is held in place and thereafter the inlet end of the perfusion tubing is attached to the source of perfusion liquid in any suitable manner.

It will be appreciated that the unbroken part/-s of the tubing providing connection between the two parts of the tubing on either side of the membrane provides for holding the membrane together, no rupture due to pulling will happen. It also ensures that no extra measure has to be taken, e.g. removing an inside support for the membrane. The probe according to the invention will not provide any hindrance to the flow through the same, while still providing the necessary flexibility to the probe while inserted in a soft and/or moving tissue.

While the present invention has been described in terms more or less specific to one preferred embodiment, it is expected that various alterations, modifications of the described embodiment will be readily apparent to those skilled in the art. The invention is thus described by the appended claims.

1. A linear microdialysis probe comprising a continuous length of flexible tubing having at least one window formed therein, said window covering at least one part of the circumference of the tubing, while the remaining part forms at least one unbroken connection between a first end of said tubing and a second end of said tubing, one of said ends adapted to be attached to an inlet for perfusion liquid and the other end forming an outlet for the dialysate, said at least one window exposing a tubular semipermeable membrane.

2. A linear microdialysis probe according to claim 1, wherein the tubular semipermeable membrane is arranged inside the flexible tubing and sealed thereto.

3. A linear microdialysis probe according to claim 2, wherein the more than one window is arranged in the flexible tubing, each such window exposing the tubular semipermeable membrane.

4. A linear microdialysis probe according to claim 3, wherein the more than one window is arranged on a circumference of the tubing adjacent to each other.

5. The linear microdialysis probe according to claim 1, wherein the window is arranged in the flexible tubing, the window exposing the tubular semipermeable membrane.

6. The linear microdialysis probe according to claim 5, wherein the at least one window includes a plurality of windows in the tubing.

7. A linear microdialysis probe, comprising:
a continuous length, flexible tube including a wall, a first end, and a second end;
at least one window formed in the wall of the tube intermediate the first end and the second end, the window extending in a circumferential direction of the tube and only extending partially around the tube;
wherein the tube includes a remaining part, circumferentially aligned with the window, that forms at least one unbroken connection between the first end of the tube and the second end of the tube,
wherein the first end is to attach to an inlet for perfusion liquid;
wherein the second end forming an outlet for the dialysate; and
a semipermeable membrane positioned within the tube and aligned with the window.

8. The probe of claim 7, wherein the membrane is tubular to substantially match the inner diameter of the tube.
